(19) United States
(12) Patent Application Publication
(10) Pub. No.: US 2005/0054015 A1
(43) Pub. Date: Mar. 10, 2005

(54) Information enhanced antibody arrays

(76) Inventor: Moncef Jendoubi, San Francisco, CA (US)

Correspondence Address: ORRICK, HERRINGTON & SUTCLIFFE, LLP, 4 PARK PLAZA, SUITE 1600, IRVINE, CA, 92614-2558, US

(21) Appl. No.: 10/945,784

(22) Filed: Sep. 20, 2004

Related U.S. Application Data
(62) Division of application No. 09/905,484, filed on Jul. 13, 2001, now abandoned.

(51) Int. Cl. G01N 33/53; G01N 33/542; C12M 1/34

(52) U.S. Cl. 435/7.9; 435/287.2; 427/2.11

(57) ABSTRACT

The present invention is directed to an array of antibodies drawn to the gene products of a cell, where the antibodies are bound to a solid support. The antibodies of the antibody array are correlated on a one-to-one basis with the gene sequences to which the individual antibodies of the array are specific. Also, methods for use of the arrays to analyze gene expression profiling and the detection of disease are described.

### RELATED INFORMATION

This application is a divisional of co-pending application Ser. No. 09/905,484 filed on Jul. 13, 2001. The priority of the prior application is expressly claimed, and the disclosure of the prior application is hereby incorporated by reference in its entirety.

### FIELD OF THE INVENTION

The present invention relates to the use of large numbers of antibodies against known and unknown expression products of gene sequences, wherein the antibodies are bound in arrays for high throughput analysis including physiological parameters, protein expression, medical, clinical, and laboratory diagnostic analysis, and gene discovery, and wherein the antibodies are correlated on a one-to-one basis to specific gene sequences and to the expression products of the gene sequences.

### BACKGROUND OF THE INVENTION

The analysis of compounds that function in biological systems can be achieved in a wide variety of methods and using several basic strategies. Traditional chemical, biochemical, spectroscopic, immunological, and other such approaches each offer advantages and disadvantages when applied to living biological systems. The practical advantage of an immunological strategy to the study of biological processes is that the immune system is capable of producing binding proteins called “antibodies” that are produced when the immune system encounters an “immunogen” or “antigen.” The antibodies are highly reactive and specific to the anitgen and are unique and powerful reagents for a wide variety of applications. As with most biological reagents, the preparation and use of antibodies requires an efficient production methodology and an application that is well suited to the analytical rationale to which the reagent will be applied. Because of the unique nature of the antigen-antibody reaction, antibodies have been used in a large number of different analytical or diagnostic methodologies to achieve a variety of analytical or diagnostic goals. Chemical or biochemical tests using antibodies are usually called “immunoassays” because of the presence of reagents derived from the immune system.

Typically, an antibody is produced to take advantage of the capability to specifically bind an antigen, usually a protein, that is known to be involved in a biological process in which a researcher is particularly interested. The antibody provides the researcher with the ability to selectively bind the antigen and to qualitatively or quantitatively detect the presence of the antigen in a sample. Furthermore, the reaction between the antibody and antigen allows the antibody to be used to physically separate the antigen from a mixture of compounds contained in a sample. Thus, when a particular interest in an antigen exists, an antibody specific to the antigen is an invaluable tool for studying the antigen because of the binding specificity. In addition to the specific antigen at interest, other molecules, compounds, or structures that share physical structure or reactivity with the antigen in an immunoassay will also be determined. In certain applications, the discovery of compounds that mimic that reactivity with an antigen is also important. Moreover, the nature and specificity of the antibody reagents that can be generated is limited only by the diversity of available antigens and certain practical limitations on antibody production.

Most antibodies are produced by immunizing an animal, usually a non-human vertebrate and frequently a mouse, rabbit, or goat, with an antigen of interest and encouraging the animal's immune system to generate antibodies specific to the antigen that may then be detected in the animal's sera. If the antigen causes a strong enough immune response, usually requiring the passage of time and a series of “booster” immunizations wherein the antigen is repeatedly injected to provide a strong immune response, the antibody can be produced in useful quantities. This process typically requires several weeks and a reasonably large investment in labor and resources. Because of the investment of time and resources required to produce antibodies to selected antigens, antibodies generally are not produced to antigens unless the significance of an antigen is recognized such that when the antibody is available for use the utility of the antibody-antigen binding is known and the resulting immunoassay is based on that known relationship.

When the significance of a protein-related antigen is known, the gene sequence that encodes the protein can usually be determined with reasonable certainty, although variations in gene expression introduce an element of uncertainty to the precise mechanisms involved. Thus, in many traditional analyses, the protein is characterized, an antibody obtained, and the gene sequence expressing the protein is determined when the biological significance of the protein in known.

As noted above, different strategies for the use of antibody-based immunoassays has led to a wide variety of applications wherein the antibody is used as a tool to characterize or locate a particular antigen. For example, an enzyme-linked immunosorbent assay (ELISA) is an assay technique that is particularly useful to diagnose antigens such as viruses, bacteria, or proteins, including antibodies, in sera. The assay uses an enzyme-linked antigen or antibody that amplifies the detection of the antibody-antigen reaction. By forming an enzyme-linked complex of the antibody-antigen pair, colorless substrate molecules are converted by the enzyme into detectable colored products that enhance the detectability of the original antibody-antigen reaction. Particularly where quantitative analysis is desired, the antigen or antibody is absorbed onto a solid surface, such as the well of a microtiter plate, so that the reaction of a particular antibody-antigen pair can be analyzed. Using any of several variations on the ELISA, a quantitative or qualitative measurement of a particular antibody-antigen interaction can be made.

In most applications of the ELISA technique, the antigen is a well-characterized protein or polypeptide fragment whose biological significance is recognized. Where the specific nature and function of the protein is known, the gene sequence that encodes the protein may or may not be known. If the nature of the antigen is not known, then the information derived from an antibody-antigen interaction in an immunoassay is limited to the information that can immediately be derived from the physical binding of the two species. The conclusion that is drawn from such interaction is simply that the antigen in question shares structural features with the antigen from which the antibody has been raised. Although this information has some utility, the ability to use antibodies of varying specificities or reactivities on a large scale has not been available to perform a high throughput analysis of samples. Even when a number of antibodies is available, the utility of those antibodies is directed primarily towards physical separation of the antigen(s) for which the antibody is specific from a sample containing other species. Thus, in most existing applications, the binding of the antibody to the antigen permits the separation of the antibody or antigen from a sample, or identifies the presence of specific antigens or antibodies in a sample when the nature of the antigen or antibody is already known; but does not, in and of itself provide new information about the physiological significance of the protein and the expression of the underlying gene sequence.

Recently, the prospect of sequencing the entire genome of an organism has led to a great hope for discovering new genes whose expression affects a variety of diseases or physiological conditions. However, given the large number of available genes, and the absence of information regarding the function of newly discovered genes, the analysis of gene function in actual physiological events is a comparatively slow process. There is a great need to generate adequate biological reagents that would allow the accomplishment of a variety of functional studies to define the physiological role of proteins and all manifestations of gene expression.

The current strategies employed to interrogate biological processes and discover new genes related to pathological conditions fail to provide a correlation between the gene expression product that may be obtained at the tissue or cellular level and the underlying gene expression. In addition, the current strategies remain costly and labor intensive. A need exists for the ability to analyze the expression products of the genes that comprise the human genome, to identify these gene products when expressed in certain defined physiologies, and to correlate the gene expression in different tissues or cell types, such as cancer cell lines, primary cell lines, and tissue specific cell lines to the underlying gene sequences. Therefore, a need exists for methods that generate and assemble information related to genes, their pattern of expression, their gene products and their derivatives, and their cellular expression at both the tissue and temporal level.

One way to track the activity and location of gene products in various tissues, disease states, and developmental stages is through the use of a selection of antibodies. Antibodies specific for a gene product can be used to demonstrate expression in disease vs. normal cells, and its pattern of expression in normal cells and tissues over time. Unfortunately, as noted above, the production of antibodies to gene products has traditionally been an arduous and time consuming task requiring injections of a protein-antigen into a vertebrate animal with a functioning humoral immune system. Thus, starting with a DNA sequence of interest, one would ligate the sequence into a protein expression plasmid for expression of the protein in a prokaryotic or eukaryotic host cell. Two problems were often encountered in recombinant protein production: mammalian proteins are often not correctly folded and modified when produced in easily cultured bacterial or yeast cells, and mammalian or other closely related eukaryotic cells are often difficult to maintain in culture. After production of the protein in the host cell, the protein would be purified from the lysed cells. Although proteins are easily purified by these methods, optimization is usually required for each protein to avoid the contamination of the desired protein with other species. After obtaining the purified protein in sufficient quantities, one would inject the protein, usually with an adjuvant, into the vertebrate to stimulate an immune response.

The expense of time and resources inherent in the above process, even if each step proceeded smoothly, could be justified because the information gained from the use of the antibody in the analytical or diagnostic techniques was justified assuming that the protein for which the antibody was specifically raised had a known biological function or significance in a physiological context. However, the human genome contains an estimated 40,000 genes and an even greater number of total transcripts and expression products. Thus, the creation of antibodies to every gene product in an organism's genome, or even a significant portion of these gene products, would be impossible in any reasonable timescale.

The discovery that the introduction of recombinant constructs containing a gene sequence could stimulate the production of antibodies to the expression product, under appropriate circumstances, alleviates some of the drawbacks to large-scale production of antibodies. For instance, as described in M. A. Barry et al., 1995, “Protection against mycoplasma infection using expression-library immunization,” Nature 377:632-635, plasmids containing a human growth hormone gene under the control of the cytomegalovirus promoter were biolistically injected into the dermal tissue of mice utilizing gold particles as a carrier. Most mice produced moderate titer antibodies to human growth hormone after one biolistic treatment, and spleenocytes isolated from the mice were used to create monoclonal antibodies to human growth hormone. However, the process of producing DNA coated microprojectiles is cumbersome and time consuming. Basically, the plasmid DNA is added to a microcentrifuge tube containing gold beads suspended in spermidine, and then precipitated onto the beads by gently vortexing with calcium chloride. After centrifuging and washing, the DNA coated beads are then mixed with ethanol and sonicated to generate a uniform gold suspension. The suspension is then transferred to a length of Tefzel tubing, coated over the interior surface, and dried. The dried DNA gold particles are then loaded into the Accell helium-powered gene delivery device for biolistic injection. Although faster than the conventional process outlined above, the gold projectile biolistic process is still too costly and labor intensive for use in generating an antibody library of a significant size and diversity.

Alternatively, highly purified plasmids containing a gene have been directly injected into the muscle of mice, as described in U.S. Pat. No. 5,589,466. However, this approach only produced a weak initial antibody response, which decreased to almost baseline levels after about five weeks. In addition, the repeated cesium chloride centrifugation protocol used to prepare the plasmids for injection is cumbersome and time consuming. Thus, the need exists for a simple, fast, economical process which produces high titer antibodies directly from a gene encoding the antigen protein.

There is also a need to produce specific antibody reagents against every expressed gene in an animal to achieve a rapid characterization of the different gene products, an analysis of their function and understanding the physiological role of these genes when expressed, especially in humans, to identify the targets for human diagnostics or therapeutics. Specifically, there is a need for an apparatus that contains an organized collection of antibodies, typically organized into an array or matrix wherein discrete samples of antibodies are rationally organized such that samples may be analyzed and the binding of compounds in the samples correlated to the individual members of the array and the gene sequence whose expression yielded the specific member of the array. Furthermore, there is a need for the ability to correlate the binding of an antigen and an antibody in a rapid, high throughput analysis to the actual polynucleotide sequence that encoded the gene product for which the antibody is specific.

There is a need to analyze thoroughly the expression products of the genome of an organism on the basis of their gene-products in different cell types, such as cancer cell lines, primary cell lines, and tissue specific cell lines. A differential screening of the gene products in these cell lines can be useful to identify specific gene-products expressed, up/or down regulated, or absent in these cell lines in normal and pathological conditions.

There is a great need to discover the genes that are associated with a variety of threatening disorder such as cardiovascular disease, cancers, inflammatory, neurological and infectious diseases to discover the underlying gene functions and their relation with these diseases. Moreover, there is a great need for a method that correlate the gene with its pattern of expression, in different cell types and tissues as well as their cellular localization.

There is a great need to generate a data bank that contains all information related to mammalian genes, their pattern of expression, gene-products and their isoforms, cellular expression at both the tissular and temporal level, and the gene sequences to which the gene products are correlated. All research institution both in the academia and in private research centers will benefit from this data bank.

There is also a need to overcome the recognized impediments to gene discovery, and to possess a readily available biological tag to allow the study of the gene function in vivo. The need also exists for antibodies to accelerate the discovery of the function of novel genes in normal and pathological conditions, to discover and design diagnostic kits to prevent or/and evaluate the progress of a particular pathology, and to discover candidates for therapy against a variety of diseases.

There is a need for method that will allow the production of substantial amount of polynucleotide templates from a large number of independent clones. This method should yield a polynucleotide template of an optimal quality that elicits a strong immune response following DNA immunization.

### SUMMARY OF THE INVENTION

The present invention takes advantage: (a) of the efficiency and the specificity and the wide immune response against a particular antigen in any form; including proteins, protein fragments, carbohydrates, organic molecules, or any immunogen against which the immune system of an animal will produce antibodies; (b) of the use of an animal with an immune system capable of mounting a distinct response against such particular antigens; (c) of the need to analyze gene expression and to discover new genes and new gene expression products, particularly proteins having physiological significance in human disease; and (d) of the need for rapid high throughput analysis of samples with a correlation between antigen present in a sample and specific gene expression, including the actual polynucleotide sequence of the gene. The present invention enables the production of antibodies against known and unknown genes products of plants, insects, pathogens, and mammals and the immobilization on a solid support, preferably in an array or matrix for high throughput analyses and gene discovery. The present invention also includes the use of a gene product array for high throughput testing by the antibodies obtained pursuant to the practice of this invention. In the method of the invention, antibody arrays are used to analyze gene product expression profiling of plants, insects, pathogens, and mammalian tissue and cell gene product extracts for high throughput analysis. The antibody arrays are used to examine gene product expression profiling of a given normal or diseased tissue, or sample from a patient with disease or suspected of disease, or for analyzing a cell type before and after exposure to drugs, chemicals, or physical stimuli, such as carcinogens, irradiation, toxic agents, pharmacological agents, and the like. Antibody arrays are also used to discover gene products related any chemical, toxic, or physical agent and/or any other stimulus.

The antibody arrays of this invention allows the examination and analysis of hundreds of gene products all at once, and reflect a specific physiological or pathological state of the sample, whether derived from the cells, tissue or biological fluid from a host. The host, for example a human patient, may be in a normal or disease state, or exposed to a stimulus or chemical or biological agent. In any case, this high throughput analysis improves current methodologies that examine only one or few gene products in any given time. For example, exposure of a patient sample to an antibody array may be useful in identifying a gene product expression profile caused by the onset of disease, the progression of disease, the resistance to treatment, or the relationship between toxic agents and abnormally regulated gene products, or virtually any pathological and physiological changes characteristic of a normal or disease state.

The present invention also includes methods for antibody screening in a multifunctional assay. The methods of the present invention can be used to identify antibodies that exert important physiological effect in plants, pathogens, insects, mammals, or in any other biological organism. The methods of the present invention identify and correlate the relationship of the antibody, the gene product and to the polynucleotide sequence (DNA and RNA) coding for the gene product that is the binding partner of the antibody. These components (antibody, gene products and polynucleotide sequences) are used to identify antibodies or functional equivalents that activate or block an important function in any targeted biological organism. Once identified by the antibody-antigen interaction, the present invention is also related to derivatives, agonists, or antagonist defined by the antibody and the gene product and their derived forms to control an important physiological or pathological pathway.

The present invention includes a method of producing high titer antibodies to a gene product encoded by a nucleic acid sequence by injecting an extracellular DNA construct encoding the gene product, and a specially formulated innoculant comprise of bacterial ribonucleic acids, into a vertebrate capable of a humoral immune response. Preferably, the vertebrate used is an outbred mouse, and the injection is made inguinally. The DNA construct is injected into the vertebrate in amounts sufficient to allow the construct to be taken up by the cells of the vertebrate so that sufficient amounts of the encoded protein are produced to induce the production of antibodies to the protein in the vertebrate. Amounts of DNA construct encoding the protein injected into the mammal are preferably in the range of about 10 to about 500 μg, more preferably in the range of about 200 to about 450 μg, and most preferably in the range of about 250 to about 400 μg. The amount of bacterial RNA injected into the vertebrate is preferably more than 1/10,000 of the amount of DNA encoding the protein, more preferably more than 1/1000 of the amount of DNA encoding the protein, and most preferably more than 1/100 of the amount of the DNA encoding the protein, as determined by weight. It is preferred that the amount of bacterial RNA be less than equal to the amount of DNA encoding the protein, as determined by weight.

In a first aspect, the devices of the present invention relate to an array of antibodies drawn to gene products, where the antibodies are bound to a solid support and wherein each antibody has a one-to-one correspondence with the underlying gene sequence. In preferred embodiments, the array of antibodies may comprise monoclonal antibodies, polyclonal antibodies, or a mixture of monoclonal and polyclonal antibodies. The array may also comprise antibody fragments, either in its entirety, or as a mixture with other antibodies. In particularly preferred embodiments, the array of antibodies is specially selected such that members of the array or collections of members of the array correspond to specific disease states such that the members of the array have a predetermined relevance to a disease state. In such arrays, collections of antibodies may correspond to markers for disease such as tumor markers in cancer patients, indicators of cell proliferation, angiogenesis, angiogenesis inhibition, vascularization of tumor or normal tissue, metastasis, apoptosis, altered cellular metabolism or differentiation, or virtually any physiological phenomenon whose manifestations can be correlated to a change in the underlying gene expression of one or more sequences and the presence or absence of one or more gene expression products in a sample.

Another aspect of the invention also relates to a method for producing polynucleotide templates comprising DNA and/or RNA for genetic immunization of an animal, preferably a rodent. Production of the polynucleotide is achieved by: (1) growing a prokaryotic cells containing a recombinant vector construct which bears the partial or complete polynucleotide sequence of any given gene; (2) lysing cells containing the recombinant vector construct to obtain a lysate; (3) treating the lysate to remove insoluble material and obtain the plasmid solution in a non pharmaceutical solution; (4) precipitating the polynucleotide templates containing DNA and RNA to recover the polynucleotide immunizing solution containing the partial or complete polynucleotide sequence of any given gene of interest. The polynucleotide template is produced without organic extractants or/and solvents. The polynucleotide templates containing the recombinant vector construct is a non-pharmaceutical grade material in a suitable solution for injection and in this formulation is specifically formulated and designed to stimulate the immune system and allow a strong immune response by the animal against the gene of interest within the plasmid.

Another aspect of the invention relates to a method for producing polynucleotide templates containing recombinant vector construct DNA and RNA suitable for genetic immunization from a microorganism and/or from any transformed cell type. The method is particularly focused on the process for the isolation and purification of hundreds of micrograms of polynucleotide templates containing DNA and RNA sequences from hundreds of different cell clones transformed with recombinant vector constructs harboring an encoding polynucleotide sequence that encodes for a partial or total gene. The polynucleotide template containing DNA and RNA is a non-pharmaceutical-grade compound in a solution specially designed as an immunostimulant. The method of the invention is useful to produce a large number of polynucleotide templates containing DNA and RNA of a partial or total gene sequence. Polynucleotide templates are used to produce specific antibodies against a gene product encoded by a partial or complete gene sequence. The method allows the production of a potent immunogenic polynucleotide template solution to immunize mammals, as evidenced by the production of specific antibodies to an expressed gene product. The immunization of mammals using polynucleotide template is an efficient and prolific method to produce antibodies and offers many advantages over classical immunization. The present invention allows (1) the production of a large number of polynucleotide templates at a very low cost, (2) preparation of several hundred of micrograms of polynucleotide template from each sample, (3) the use of a potent polynucleotide template into a mammal to produce specific polyclonal antibodies within a relatively short period of time comparatively to classical immunization protocols, (4) the production of specific polyclonal antibodies with a single injection of immunizing template.

### DETAILED DESCRIPTION OF THE INVENTION

The term “antibody” refers to an antibody (e.g., a monoclonal or polyclonal antibody), or antibody fragment, having specific binding affinity to a gene product of a cell or a fragment of the gene product. The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target. The antibody molecule is a glycoprotein comprising at least two light polypeptide chains and two heavy polypeptide chains, wherein each light and heavy chain contains a variable region located at the amino terminal portion of a polypeptide chain featuring an antigen-interaction region wherein the antigen is bound. The heavy and light polypeptide chains are also comprised of a constant region at the carboxy terminal portion.

By “an array of antibodies” it is meant a group of antibodies bound to a solid support, where at least two of the antibodies in the array are directed to different binding partners. The antibodies are preferably arranged to form a line. However, the antibodies may also be arranged in any other formation, such as in a circle, a semi-circle, or to form shapes, such as 5, ?, or +, or any other shape.

“Gene product” or “gene expression product” comprise the broad class of compounds that are produced by translation or transcription of a gene in a cell. These products include peptides, such as, but not limited to, proteins, polypeptides, and oligopeptides, or nucleic acids, such as, but not limited to, RNA, including messenger RNA, transfer RNA, ribozomal RNA, and DNA. The proteins include full-length proteins exhibiting normal folding patterns as well as fragments and other polypeptides modified by translational or post-translational processing including fragmentation, glycocellation, and other known phenomena. Any polynucleotide or oligonucleotide, whether comprising a ribose or a deoxyribose, is within the definition of “gene product” or “gene expression product.”

The terms “genetic immunization” mean the injection of polynucleotide materials operatively expressible in mammalian tissue upon injection. The expressed polynucleotide material will produce a gene product against which a specific antibody is made by the immune system of the mammal in a sufficient quantity to be detectable and useable in forming the arrays of the present invention and has sufficient specificity to bind gene products as described herein.

The term “kit” refers to assemblies of diagnostic apparatus for performing such methods may be constructed to include a first structure containing the antibody or array of antibodies, as defined herein, and a second container having a conjugate of a binding partner of the antibody and a label, such as, for example, a radioisotope. The diagnostic kit may also include notification of an FDA approved use and instructions therefore including specific information correlating each member of the antibody array to the gene expression product and/or the polynucleotide sequence of the underlying gene. Moreover, the kit may contain specific indicators identifying the significance of the presence or absence of a binding event when a sample is exposed to the array and a measurable signal or detection of the antibody-antigen binding event is present. The kit may also include means such as an index or key pursuant to which each member of the array may be correlated to a polynucleotide sequence that exists in a separate record or is part of a data base that is correlated to the specific members of the array, or a collection of members of the array. In particularly preferred embodiments, the specific antibodies and their corresponding polynucleotide sequences are correlated to the presence or absence of a disease state.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art. See, for example, Kohler, et al., Nature 256:495-497 (1975), and U.S. Pat. No. 4,376,110.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

The term “polynucleotide templates” means a mixture of polynucleotide sequences including RNA and DNA of different sizes and conformations, produced by different cell types of eukaryotic and prokaryotic origin. These sequences may be expressible in prokaryotic systems or in mammalian systems and may include their components as described herein.

A “polypeptide” refers to a molecule comprising ten or more amino acids, linked together in at least one chain through amide, or peptide, bonds. An “oligopeptide” is similar to a polypeptide, except that it comprises less than ten amino acids. Similarly, a “polynucleotide” is a molecule comprising ten or more nucleotides, linked together in at least one change through phosphodiester bonds. An “oligonucleotide” is similar to a polynucleotide, except that it comprises less than ten nucleotides.

The term “recombinant vector construct” means a composition of plasmid origin, viral origin, or a combination of both. Recombinant vector constructs have regulatory elements such as enhancers, promoters, kozak sequences, polyadenylation signals and the like and allow operative expression of a cDNA in mammalian cell type or tissue. Recombinant vector constructs may possess a sequence for the origin of replication in prokaryotic system and a gene that encodes for an antibiotic selection to confer resistance for the transformed organism with the recombinant vector construct. Recombinant vector constructs may contain sequences that facilitate integration upon its penetration into a mammalian cell or injection into a mammalian tissue in vivo.

The “solid support” on which the antibodies are bound is any solid support capable of being used in biological or biochemical testing. Examples, without limitation, of solid support include glass, silica, silica gel, silicon wafer, silicone, plastics, such as those made of polyethylene, polystyrene, polyvinyl chloride (PVC), or polyvinyl pyrrolidone (PVP), nylon, TEFLON®, nitrocellulose, ceramic, fiber optic, semiconductor material, etc.

By “specific binding affinity” is meant that the antibody binds to target polypeptides with greater affinity than it binds to other polypeptides under specified conditions. Antibodies having specific binding affinity to a gene product may be used in methods for detecting the presence and/or amount of the gene product in a sample by contacting the sample with the antibody under conditions such that an immunocomplex forms and detecting the presence and/or amount of the antibody conjugated to the gene product.

The cells, tissue samples, biological fluids or derivatives or extracts of these that are used in the invention are from an organism, either a single cell organism, such as bacteria, viruses, amoebae, or protozoa, or multicellular organisms, such as members of the plant and animal kingdoms. The organism is preferably a plant, an insect, or an animal. The plant is preferably selected from the group consisting of crops, such as grains, nuts, vegetables, and fruits, household plants, trees, and bushes. In most diagnostic applications, the sample is from a human patient and may include tissue from a normal or disease state such as a tumor, a biological fluid such as ascites, urine, plasma, serum, spinal or cerebral fluid, or other preparation and may be processed for advantageous use in the kits of the invention.

In another aspect, the invention is directed towards a method of analyzing gene expression in an organism, comprising the steps of:
a) producing antibodies against known or unknown gene products of an organism;
b) creating an array of the antibodies on a solid support;
c) contacting samples containing proteins from the organism with the array of antibodies so that the gene products bind to their respective antibodies in the array, where the binding can be measured;
d) determining the presence or absence, and if present, optionally determining the amount of, the gene products bound to the antibodies; and
e) correlating the result of the determination step (d) to the expression of at least one gene, wherein the polynucleotide sequence is known.

In preferred embodiments, the organism is a human patient and the gene products relate to a certain condition. The certain condition is preferably selected from the group consisting of suspicion of or screening for disease, progress of disease, or disease resistance, or response or resistance to treatment is known. The gene products may be obtained from the organism from any source tissue or sample as described herein.

In another aspect, the invention relates to a method of diagnosing a specific disease in an organism, comprising the steps of:
a) selecting antibodies against gene products known to correlate to a disease affecting the organism;
b) creating an array of the antibodies on a solid support;
c) contacting a sample from the organism to the array of antibodies so that the gene products bind to their respective antibodies in the array, where the binding can be measured;
d) determining the presence or absence, and if present, the amount of, the gene products bound to said antibodies; and
e) correlating the results to the presence or absence of disease.

In another specific aspect of the above invention, a method of diagnosing a disease in an organism is comprised .the steps of:
a) producing antibodies against gene products of the organism, where the gene products are expressed in response to infection by a pathogen;
b) creating an array of the antibodies on a solid support;
c) contacting cell lysates from the organism to the array of antibodies so that the gene products bind to their respective antibodies in the array, where the binding can be measured;
d) determining the presence or absence, and if present, the amount of, the gene products bound to the antibodies; and
e) compare the result of the determination step (d) to a standard.

Preferably, the organism is a human patient and the pathogen is selected from the group consisting of amoebae, fungi, viruses, and bacteria.

### EXAMPLE 1

### DNA Preparation Protocols—Existing techniques and modifications of the present invention

Laboratory protocols and techniques to produce and prepare DNA or RNA materials from any organism, mammal, pathogen plant or others are well known to those of skill in the art. There are several alternative methods to prepare and purify DNA or RNA materials are described in detail in Current Protocol in Molecular Biology, Volume 1, edited by Frederick M. Ausubel, 1996. In most conventional techniques, polynucleotide materials, DNA and RNA, are required to be highly pure to correlate the data to the effect of the polynucleotide materials and to avoid contaminating material in the polynucleotide solution that could interfere with the immunization. In these techniques, the process of polynucleotide purification requires several steps.

Current laboratory methods to prepare plasmid DNA templates require extensive investments of time and resources. There are two widely used laboratory procedures for the preparation of lysate solution containing plasmid DNA: the boiling method and the alkaline lysis method. Both methods utilize laboratory scale centrifugation to separate cellular debris from the crude lysate. Organic extraction with phenol/chloroform/isoamyl alcohol or a variation of this mixture is typically used to improve the purification of the plasmid DNA template. Further purification of plasmid DNA template is improved by laboratory scale ultracentrifugation using cesium chloride and ethidium bromide for generally more than 15 hours, followed by several butanol extraction and dialysis for 48 hours. Because this procedure is a labor intensive manipulation, it cannot be used to purify a large number of plasmid DNA template at the same time.

The method described above for obtaining purified DNA template is not an optimal protocol. Organic solvents are problematic. These chemicals are highly toxic and add significant expense to the method in terms of not only storage, safe use and disposal of hazardous waste but also procedures for validation of their removal. The ethidium bromide is highly mutagenic and teratogenic reagent and present a significant problems of safe disposal. The administration of DNA solution to a mammal even with traces of ethidium bromide could be harmful.

In variations of the methodology described above the crude lysate is treated with pancreatic RNAse followed by alkalin detergent treatment to reduce the presence of bacterial RNA. An organic extraction with phenol/chloroform is followed by ethanol precipitation, resuspension and a second ethanol precipitation. This procedure of template DNA purification remains a time consuming process that limit its utilization to produce a large number of DNA templates. Alternatively, alkaline solution lysate of a crude cell extract is centrifuged to remove cell debris and the remaining solution is treated to precipitate the polynucleotide templates. The polynucleotide templates is resuspended in a Tris-HCl/EDTA buffer and passed through an exchange column for further purification. Again, this method remains a laboratory scale and time consuming and limited in scope.

Thus, the state of the prior art requires that DNA immunization can be achieved only with high pure DNA template. A such requirement imposes a series of technical manipulation to obtain the required quality of pure DNA, and prevents the preparation of large number of polynucleotide templates in a reasonable time and with reasonable costs.

In a preferred embodiment of the present invention, the process and the method described herein is rapid, economical, scalable to large number of gene sequences and suitable to purify large numbers of polynucleotide templates for genetic immunization.

To produce the antibodies used in the array, a polynucleotide template is prepared with an expressible polynucleotide coding for an immunogenic translation product that is introduced into a vertebrate wherein the translation product is formed to elicit an immune response against the gene product.

The polynucleotide template used for immunization pursuant to this invention is preferably DNA and/or RNA sequences, although a non-replicating, replicating recombinant DNA sequence or an integrating may be used. The preparation of nucleotide template of different fragment of the same gene sequence may be used to perform genetic immunization with the aim of making monoclonal antibodies. Typically, a gene sequence encoding for a gene product is divided into small fragments of 20, 30, 40, 50, 60, 70, 80, 90 and 100 base pairs or any integral value in any of these ranges or any interval of 10 or 100, preferably between 20 and 1000, but possible between 1000 and 5000, or more. These fragments may or may not overlap between themselves. The polynucleotide template may be introduced into tissues of the body using the injectable carrier alone; liposomal preparations are preferred for methods in which in vitro transfections of cells obtained from the vertebrate are carried out. The carrier preferably is isotonic, hypotonic, or weakly hypertonic, and has a relatively low ionic strength, such as provided by a sucrose solution. The preparation may further advantageously comprise a source of a cytokine which is incorporated into liposomes in the form of a polypeptide or as a polynucleotide translatable into gene product. The polynucleotide immunization selectively elicits a humoral immune response, a cellular immune response, or a mixture of these. In embodiments wherein the cell expresses major histocompatibility complex of Class I, and the immunogenic peptide is presented in the context of the Class I complex, the immune response is cellular and comprises the production of cytotoxic T-cells.

The polynucleotide templates may include prokaryotic and eukaryotic recombinant vector constructs. Potentially, recombinant vector constructs are derived from plasmid and RNA and DNA viral origin or a combination of both. Recombinant vector constructs may incorporate various origins of replication for both eukaryotic and prokaryotic systems. These vectors can also encompass a number of genetic elements to facilitate cloning and expression of selectable genes and/or polylinkers, promoters enhancers, leader peptide sequences and alike. The selection of vectors, origins, and genetic elements may vary according to need and the host cell, and is within the skill of workers in this art. A host cell can be among the following cells but not limited to them: bacteria, yeast, fungi, insect and mammalian cells. Preferred cells are microbial cells particularly E. coli. Any suitable strain of E. coli is contemplated in this invention. Likewise genes encoding diverse structural proteins or peptides, polypeptides, glycoproteins, phosphoproteins, etc, are also contemplated in the present invention. The inserted cDNA encoding gene product into the recombinant vector constructs may correspond to partial or complete cDNA, to genomic DNA fragment, to synthetic DNA, to polynucleotide sequences from any of the following genomes: mammalian genomes, pathogen genomes, plant and insect genomes, but not limited to these referred to genomes. These sequences may be obtained by using chemical synthesis or gene manipulation techniques.

The polynucleotide template for vertebrate immunization contains an operatively coding recombinant vector construct for a partial or a complete gene product. The polynucleotide operatively codes for a gene product and has all the genetic information necessary for expression, such as a suitable promoter, enhancer, polyadenylation signal and the like. The polynucleotides may be partial or complete sequences of any gene. These polynucleotides can be administered to a mammal by any method that delivers injectable or inhalation spray materials to the components of the immune system of a mammal, such as by injection into the interstitial space of tissues such as muscle or skin, introduction into the circulation or into body cavities or by inhalation or insufflation, or injection into any tissue of the mammal that is exposed to the immune response. The pH of the preparation of the delivered material is suitably adjusted to physiological pH ranges.

The polynucleotide templates may or may not integrate in the recipient cell genome and the template may contain integration-facilitating constructs, changed lipids, viral particles, or other such compounds. These polynucleotide templates may be non replicating DNA sequences, or may have been genetically engineered to possess specific replicating elements to insure the maintenance and replication of the desired delivered polynucleotide templates. These features will allow the production of the desired gene product material for extended periods. The polynucleotide sequences to be administered to the immune system of the mammal may be prepared from eukaryotic expression DNA libraries, referred to as cDNA, derived from particular tissue and/or cells of a mammal.

The cDNA is preferably driven by strong eukaryotic promoter such as RSV, LTR, CMV, ACTIN, PGK and other specific or non specific tissue promoter and an appropriate regulatory element to achieve the highest possible expression of the administered cDNA in eukaryotic cells and or in mammalian tissues. Typically, these expression cDNA libraries are enriched cDNA libraries for partial and/or complete gene sequences that reflect the desired physiological or pathological state. The process of the construction of an enriched cDNA leads to a removal and/or reduction of common gene sequences present in both normal and disease cell types, to maximize the enrichment of specific gene sequences for the tissue expressed preferentially or solely in the disease state. Methods to construct enriched or subtractive libraries with a desired intent are well known by skilled individuals in this art. Such methods are extensively described in molecular biology manuals such as the Current Protocols In Molecular Biology John Wiley, edited by Frederick M. Ausubel et al., 1996, and according to methods for molecular biology described in the Manual For Molecular Biology, T. Maniatis, Cold Spring Harbor, 1996. Alternatively, cDNA libraries may be purchased through a commercial vender. Enriched expressed cDNA libraries may be made from brain, lung, heart, liver, ovary, testis, spleen, and other tissues. These expressed cDNA libraries are plated pursuant to conventional methods, on a solid LB agar medium containing an antibiotic for selection of bacteria that bear a vector construct. The recombinant vector construct contains also a particular cDNA that could correspond to a full length or to a partial coding sequence of a given gene.

Transformed eukaryotic or prokaryotic cells with recombinant vector construct containing cDNA libraries from enriched or non enriched cDNA libraries may be plated on solid agar LB medium or any other medium. A such medium contains preferably an antibiotic to select only transformed cells with recombinant vector construct containing cDNA sequences. Preferably transformed cells with the recombinant vector construct containing cDNA sequences are bacteria. Resistant clones are picked up individually and transferred to 96 well plates containing the LB medium with the same antibiotic for selective growth. 96 well plates containing transformed bacterial clones are grown overnight and duplicated in two 96 well plates. The first 96 well plate receives 100 microliters of 80% glycerol in each well and frozen at −80° C. for further usage. Each clone form the second 96 well plate is transferred to 12 multiwell plate containing 3 ml of LB medium and grown overnight to prepare polynucleotide templates. Other multiwell plates or individual tubes may be used to grow up the transformed clones for polynucleotide template preparation.

Typically, selected bacterial clones containing recombinant construct bearing a cDNA sequence are grown for 16 hours, harvested by centrifugation, lysed using alkaline buffer and chromosomal bacterial DNA and protein are then precipitated by adding potassium acetate. A glass fiber disc is overlayed on the solution and centrifuged for 15 minutes at 3000 rpm using a swinging rotor. A forceps is applied to purify the polynucleotide templates from the precipitate of the cellular proteins and chromosomal DNA. Alternatively, isopropanol (0.7 of the total volume of the lysate solution) is added to precipitate the polynucleotide material by centrifugation using a swinging rotor. The supernatant is discarded and polynucleotide template recovered from the plastic using 300 microliters of Tris-HCl 10 mM, EDTA 1 mM. Alternatively, individual bacterial clones containing cDNA recombinant may be grown, harvested, lyzed, and polynucleotide templates precipitated using a 96 well plate.

### EXAMPLE 2

### (Polynucleotide DNA or RNA) Immunization—Existing techniques and modifications of the present invention.

DNA material has been used to induce a protective immune response in a mammal by injecting a DNA sequence in a non-integrating composition. See U.S. Pat. No. 5,589,466. In addition antibodies were generated through lipid mediated DNA delivery Pat. No. 5,703,055. In these cases as well as in other experiments related to the use of DNA as a basis for genetic immunization, the polynucleotide material is highly pure and the requirement of purity is emphasized in the literature. In fact, the purity requirement has been stressed to be a mandatory requirement for the expression of gene of interest into the mammal and a successful genetic immunization. As described above, the high quality DNA requires a stringent purification protocol having several purification steps and requiring centrifugation of the DNA template. The purification procedures effectively preclude the efficient preparation of a large number of DNA templates for genetic immunization.

Another method of DNA immunization uses a gene gun to deliver the plasmid DNA to a mammal. This method delivers polynucleotide complexed with gold particules to mammals to achieve DNA vaccines. Basically, polynucleotides are added to a microfuge tube containing gold beads suspended in 100 mM spermidine. While gently vortexing the tube, a solution of calcium chloride is added to precipitate the DNA onto the beads, and the tube is allowed to stand for approximately 10 minutes to complete the precipitation. The DNA coated beads (2 microgram of polynucleotide per microgram of gold) are pelleted and the supernatant removed. The gold/DNA pellets are washed twice by vortexing in ethanol and centrifuging. The DNA/gold beads are then transferred to new tube, mixed with ethanol and sonicated to generate a uniform gold suspension. Using a syringe attached by an adapter, this suspension is drawn into a 30 inch length of Tefzel tubing to yield a 1 milligram gold/DNA per inch of tubing. The tubing is then transferred into a tube turner. After allowing the beads gold to settle the ethanol is slowly drawn off, and the turner rotated for 30 seconds, smearing the gold/DNA around the inside of the tubing. The DNA gold particules are dried and loaded into a helium-powered gene delivery device.

Pursuant to this invention, non pharmaceutical-grade polynucleotide templates containing DNA or RNA or both from an organism of choice are formulated in a solution that is physiologically acceptable for genetic immunization of a mammal, but specially designed and formulated to produce a strong immunological reaction against the gene product encoded by the polynucleotide template. This process produces a recombinant vector construct composed of supercoiled, concatemeric, and relaxed DNA. The limited purification procedure is free of solvent, organic, or mutagenic solutions and involves limited manipulation. The method of the invention can be scalable and with the performance of limited DNA prep, can prepare hundreds of polynucleotide templates simultaneously using limited and disposable materials. This process of polynucleotide preparation allows the production of large number of polynucleotide templates (at least several hundred) from different clones and substantial quantities of polynucleotide templates (hundreds of micrograms, milligrams) from each clone. In contrast to other methods known in the prior art of DNA immunization, the method of the invention requires a single tube or a single well plate for the preparation of a polynucleotide template. In practical application, several thousand polynucleotide templates containing plasmid DNA can be prepared every day based on this invention. Finally, the present invention is substantially more economical than current methods, and is suited to economic preparation of a large number of polynucleotide templates for genetic immunization. Thus, the present invention uses a simple, economical, rapid, reliable and reproducible method to prepare a high yield of polynucleotide templates comprising recombinant vector construct, DNA and RNA sequences.

The preferred methodology uses a high-throughput preparation method to yield a large number of polynucleotide templates containing DNA and RNA from prokaryotic and eukaryotic systems for genetic immunization. The polynucleotide templates contain a recombinant vector construct that harbors a partial or complete gene sequence of any gene. The gene, whether partial or complete, is derived from a plant, insect, pathogen, mammal, vertebrate, invertebrate or any other organism. The delivery of the polynucleotide materials, prepared by the present methodology, yields a high efficiency immune response yielding specific polyclonal antibodies against one or more, but preferably one, gene product encoded by the corresponding gene sequence. The gene sequence is present in the polynucleotide template and it is preferably under the control of appropriate regulatory elements.

The polynucleotide template is prepared from recombinant cells, either eukaryotic or prokaryotic, and including but not limited to bacteria such as E. coli, yeast, mammalian and insect cells. Pursuant to the present invention, the desired recombinant organism is grown in a multiwell container for approximately 12-16 hours. Organisms are harvested by centrifugation and the culture medium is discarded. The microorganisms are lysed gently and the polynucleotide template is purified in one step using a glass fiber to remove cellular precipitated material. Glass fiber is overlayed on each sample and centrifuged. Then, forceps are applied to remove the glass fiber, the bacterial precipitated protein, and chromosomal DNA, leaving the polynucleotide solution ready for genetic immunization. Alternatively, the polynucleotide template may be concentrated by adding isopropanol, followed by centrifugation. The gene product may be modified or administered with an adjuvant in order to increase its antigenicity. Methods of increasing the antigenicity of a gene product are well known in the art.

In another embodiment, the application of glass fiber to the lysate solution of transformed bacteria may be replaced by other equivalent materials such as nitrocellulose, whatmann paper, agar, cellulose, ceramic or other materials. These materials may be applied to purify the polynucleotide solution from cell debris and chromosomal DNA following centrifugation. Typically transformed bacterias are grown individually, harvested, lysed in using 0.1% SDS, Tris-HCl 50 mM, pH 8, 20% glucose, 150 mM NaCl and 10 mM EDTA. Alternatively, transformed bacterias are harvested and killed using a chemical solution such as 2% gluturaldehyde, or other chemical or physical means. Killed bacteria are then solubilized in Tris-HCl 50 mM, pH 8, 150 mM NaCl and 10 mM EDTA and delivered to a tissue of a mammal.

In a preferred embodiment of the invention, bacterial clones are grown individually for each polynucleotide template and are prepared as described above for genetic immunization of a single mouse per selected polynucleotide. Five weeks after the first immunization, animals are bled and sera are used to initially test the presence of antibodies against the gene product encoded by the corresponding gene. When the immune reaction is satisfactory, animals are sacrificed and their spleen removed. Splenocytes are separated from the rest of cells and connective tissues and fused with myeloma cell lines such as Sp2/0. Hybrid clones are grown in an appropriate selection medium for 10 to 14 days and their medium supernatant is tested for the production of specific polyclonal antibodies against the corresponding gene product.

The gene products used in the methods of the present invention can be used to produce polyclonal antibodies as the direct result of DNA immunization, or, with further processing of the animal spleen, for the production of hybridomas. A hybridoma is an immortalized cell line which is capable of secreting a specific monoclonal antibody. Thus, the antibodies of the present invention include monoclonal and polyclonal antibodies, as well as fragments of these antibodies, and humanized forms. Humanized forms of the antibodies of the present invention may be generated using one of the procedures known in the art such as chimerization or CDR grafting. In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods 35:1-21, 1980, which is incorporated by reference herein, including any drawings). In a preferred technique, for the production of monoclonal antibodies, spleen cells from the immunized animals are removed, fused with myeloma cells, such as SP2/0-Agl4 myeloma cells, and allowed to become monoclonal antibody producing hybridoma cells. Any one of a number of methods well known in the art can be used to identify the hybridoma cell, which produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art (Campbell, “Monoclonal Antibody Technology: Laboratory Techniques in Biochemistry and Molecular Biology”, supra, 1984).

For polyclonal antibodies, antisera containing the antibodies is isolated from the immunized animal and is screened for the presence of antibodies with the desired specificity for the gene product of the corresponding polyclonal antibody using one of the above-described procedures. The above-described antibodies are preferably labeled for detection. Antibodies can be labeled through the use of radioisotopes, enzymatic labels (such as horseradish peroxidase, alkaline phosphatase, and the like) chromophore labels (such as FITC or rhodamine, biotin and the like), paramagnetic atoms, and the like. Procedures for accomplishing such labeling are well-known in the art, for example, see (Stemberger et al., J. Histochem. Cytochem. 18:315, 1970; Bayer et al., Meth. Enzym. 62:308-, 1979; Engval et al., Immunol. 109:129-, 1972; Goding, J. Immunol. Meth. 13:215-, 1976).

One skilled in the art can also readily adapt currently available procedures with regard to antibodies, to generate peptides capable of binding to a specific peptide sequence in order to generate rationally designed antipeptide peptides (Hurby et al., “Application of Synthetic Peptides: Antisense Peptides”, In Synthetic Peptides, A User's Guide, W. H. Freeman, N Y, pp. 289-307, 199; Kaspczak et al., Biochemistry 28:9230-9238, 1989).

### EXAMPLE 3

### The antibody arrays of the invention enhance the information derived from the antibody-antigen interaction.

As noted above, an important feature of the present invention is that the antibodies produced by the claimed methods are directly linked on a one-to-one basis with the DNA or DNA sequence encoding the protein to which the antibody is specific. Thus, using these methods, antibodies may be generated to known or unknown proteins encoded by known or unknown DNA sequences. By observing the reaction between the antibodies and gene expression products in a sample, the antibodies provided by this invention can be used to explore the function of known or unknown proteins and these functions and compounds correlated to the gene sequence.

For example, the expression of a gene encoding an unknown protein may be studied in normal versus cancer cells, using the polyclonal antibodies generated through the methods of this invention, in immunoassays with normal and cancer cell protein extracts. When antibodies are generated to a larger number of gene products, as applicant illustrates herein, the library of antibodies can be rationally organized and tested against samples from known sources to characterize differential expression of a multitude of individual proteins in, for example, normal versus diseased cells. The reaction of the proteins with the individual members of a rationally organized antibody array also leads to identification of a large number of gene products that are differentially expressed in the normal versus diseased state. Similarly, differential gene product expression in various cell types, tissue types, and developmental stages can be analyzed as well as any distinction between two physiological states differentiated by virtually any stimulus.

When a polyclonal sera with a particular reactivity of interest, such as reactivity to a cancer-specific protein is identified, monoclonal antibodies may be produced that display the reactivity of interest. In the monoclonal antibody-based embodiments of the method, a vertebrate, preferably a mouse, is injected with a polynucleotide construct harbouring the gene of interest as described above. Four to five weeks after immunization, animals are bled and the serum is used to test the presence of antibodies against the relevant gene product. The spleen is harvested from the vertebrate when sufficient antibodies displaying the relevant reactivity have been produced (when the immune response is judged satisfactory). Generally, high antibody titers occur within four weeks of injecting the vertebrate: thus, the spleen may be advantageously harvested within four weeks. Optionally, the spleen may be harvested at five, six, seven weeks, or later, depending on the particular need for monoclonal antibodies. Monoclonal antibodies are then prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., (1981) pp. 563-681). In general, the splenocytes are extracted, harvested, and fused with a suitable myeloma cell line. Any suitable myeloma cell line may be employed in the present invention, preferably the parent myeloma cell line (SP2/O), available from the American Type Culture Collection, Rockville, Md. After fusion, the resulting hybridoma cells are selectively maintained in appropriate selection medium (HAT), and then cloned by limiting dilution. After 10-14 days, the hybridoma medium supernatant obtained through such a selection are then assayed to identify clones which secrete antibodies which display the activity of interest. Once the monoclonal antibodies are obtained, they are used in the arrays in the same manner as the polyclonals directly resulting form DNA immunization as described herein. As with the arrays comprised at polyclonal antibodies, reaction of the monoclonal antibody and gene product yield specific information about the underlying gene expression.

Additionally, anti-idiotypic antibodies which mimic the epitopes of the protein encoded by the DNA construct may be produced utilizing the antibodies supplied by the methods of the present invention. This use of the antibodies produced by the invention takes advantage of the fact that antibodies are themselves antigens, and, therefore, stimulate the production of antibodies which bind to themselves. To produce an anti-idiotypic antibody which mimics the DNA construct encoded protein, the DNA construct encoded protein specific antibodies produced according to the methods of the invention are used to immunize a vertebrate, preferably a mouse. The splenocytes of the vertebrate are then used to produce hybridoma cells. The hybridoma cells are then screened to identify clones which produce an antibody which is able to bind to the DNA construct encoded protein specific antibodies, but which does not bind to non-specific antibodies. Such anti-idiotypic antibodies can be competitive inhibitors of the protein encoded by the DNA construct.

In addition, the antibodies produced by the methods and systems of the present invention may be labeled for use in a diagnostic assay or array as described below. Examples of suitable enzyme labels for use in ELISA systems include malate dehydrogenase, staphylococcal nuclease, delta-5 steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase. Examples of chemiluminescent labels include a luminal label, an isoluminal label, an aromatic acridinium ester label, an imidazole label, an acridinium salt label, an oxalate ester label, a luciferin label, a luciferase label, and an aequorin label. Examples of suitable fluorescent labels include an ¹⁵² Eu label, a fluorescein label, an isothiocyanate label, a rhodamine label, a phycoerythrinl label, a physocyanin label, an allophycocyanin label, an o-phthaldehyde label, and a fluorescamine label. Examples of suitable radioisotopic labels include ³ H, ¹¹¹ In, ¹²⁵ I, ¹³¹ I, ³² P, ³⁵ S, ¹⁴ C, ⁵¹ Cr, ⁵⁷ To, ⁵⁸ Co., ⁵⁹ Fe, ⁷⁵ Se, ¹⁵² Eu, ⁹⁰ Y, ⁶⁷ Cu, ²¹⁷ Ci, ²¹¹ At, ²¹² Pb, ⁴⁷ SC, ¹⁰⁹ Pd, etc.

To construct the information-enhanced arrays of the present invention, the antibodies prepared by the above-described techniques are immobilized in a rationally organized matrix on a solid support. Examples of such solid supports include, but are not limited to, plastics such as polycarbonate, complex carbohydrates such as agarose and sepharose, acrylic resins and such as polyacrylamide and latex beads. The solid support may also include glass, silica, silica gel, silicon wafer, silicone, plastics such as polyethylene, polystyrene, polyvinyl chloride (PVC), or polyvinyl pyrrolidone (PVP), nylon, TEFLON®, nitrocellulose, ceramic, fiber optic, and semiconductor materials. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974). The terms “rationally organized” mean that the antibodies are arranged in such a faction that a reaction site is created such that when a protein sample is brought into contact with a discrete quantity of the antibody organized in an array, that binding reaction at the reaction site can be detected and analyzed to correlate the specific antibody to the gene sequence used to obtain the expression product for which the antibody is specific. Thus, the rational organization of the array provides the ability to correlate the reaction of the antibody and the antigen present in the sample to the specific sequence, thus enabling analysis of differential quantities of protein contained in a sample and correlation to the underlying gene sequence expression.

The antibody array comprises a group of antibodies, which group comprises at least two antibodies and preferably comprises a much larger number including values between 10 and 100 and integral values therein, values between 100 and 1,000, in integral values therein, as well as intervals of 10, values between 1,000 and 10,000, as well as integral values therein, and intervals of 10 or 100, and values between 10,000 to 100,000, and as high as an antibody to each expression product in vertebrate, plant, or insect genomes, as well as integral values therein in intervals of 10, 100, 1,000, and 10,000. Although each individual sample or aliquot of antibodies that forms the members of the individual array can contain an antibody of more than one specificity, it is preferred that each member or dot of the array contain antibodies of a single specificity. However, a single dot or member of the array may contain antibodies with more than one specificity. In preferred embodiments of the invention therefore, at least two of the antibodies are different and in preferred embodiments at least 10, or at least 100, or at least 1,000, or at least 10,000, or at least 100,000 of the antibodies have different specficities, and are reactive with different gene products. As above, all integral values and intervals between these ranges are expressly disclosed herein. Furthermore, any numerical value in the above ranges corresponds to the number of antibodies that are coupled to a solid support to form the particular arrangement of the array. The antibodies may be arranged in a number of different ways on the solid support. For example, they may be arranged to form a line, a semicircle, a circle, a 5, a ?, or a +, or any other shape or combination of shapes. Preferably, duplicate quantities of the antibodies are arranged in a side-by-side fashion in the array to provide for reproducibility and a control. For purposes of diagnostics, the arrays may be organized on a physiological basis such that individual portions of the arrays reflect gene sequences having differential expression in different disease states. For example, an array may be constructed having gene expression products for a variety of diseases, wherein such expression products are known to be expressed in a biological fluid such as saliva, urine, serum, plasma, ascites, spinal or cerebral spinal fluid, etc. In such a rational organization, the location of binding events from proteins in a sample provides specific information about the physiological state of the underlying organism.

To prepare an array, working dilutions of source antibodies are made at 1:100 in tris buffered saline (TBS) containing 0.02% BSA, 0.02% sodium azide. Aliquots of the antibody solution are arrayed on wet nitrocellulose on 2 layers of blotting paper. Two layers of precut blotting paper are placed in Omni-tray and are soaked with TBS solution. A precut sheet of nitrocellulose is placed over the nitrocellulose. Excess wetting solution is drained by tilting it and excess liquid absorbed. The individual members of the arrays may be deposited manually or with a Robotic system (Genomic Solutions Flexys ^{7m}) to construct the array. The arrayed blots are dried on blotting papers for 5 minutes at room temperature. The dry blots are placed in 0.3% hydrogen peroxide in TBS and rocked for 5 minutes on a vertical rotor. The blots are rinsed twice in TBS solution with rocking (2 rinses of 5 minutes each). The blots are treated with blocking buffer in blocking solution (2.5% Non Fat Dry Milk in TBST) in a wide tray for 1 hour with constant rocking at room temperature. After blocking, the blots are given 2 quick rinses with TBS solution for 2 minutes each.

### EXAMPLE 4

### Gene expression profiling using the antibody array.

It is known in the art that in response to a condition or a disease, differential expression at specific genes of an organism occurs, giving rise to the presence of specific gene products in the organism's cells. For example, if an organism suffers from a viral or a bacterial infection, to combat or cope with the infection, the organism produces certain gene products. It is also known that the organism may produce the gene products specific to the condition before the organism itself shows any morphological signs of suffering. By way of example only, a person suffering from the common cold will produce specific gene products associated with the disease before the person notices a runny nose or watery eyes.

Similarly, in carcinomas, the up or down regulation of genes or gene products that cause or accompany the disease state will result in the differential expression of genes and the differential presence of gene products in a sample. The gene products contained in virtually any biological sample may be described as “cell contents” because such products are excreted, derived, or extracted from a cell source such as tissue, plasma, tumor cells or tissue, etc. To test whether an organism is suffering from that disease or condition, the cell contents are exposed to the antibody array. The binding events at the reaction sites of the antibody array enables the identification of the gene products excreted, derived, or extracted from the cell. If the gene products related to the disease or condition are either present or absent, as determined by the binding of the products to the array, then it becomes apparent that the organism is suffering from the disease or condition.

A test solution of biotinylated human serum (B-HS) and biotinylated blucose-6-phosphate dehydrogenase (B-G6PDH) is prepared in 10 ml volume per blot. For example, 100 ul B-Hs of 4 mg/ml stock concentration and 5 ul of 10 ug/ml stock solution of B-G6PDH are added in 10 ml of pre-mixed solution of 80:20 TBS blocking buffer, mixed thoroughly, and sonicated at high frequency for 20 minute at room temperature. The sonicated solution is added to the blot in an Omni-tray. The solution is first poured in the Omni tray (10 ml) and thoroughly spread in the Omni tray, and then the blocked and washed nitro-cellulose blots are placed therein. Each blot is incubated individually with the test solution for 1 hour at room temperature on the rocker. After incubating, the gene product sample solution is poured onto the array and rinsed with 10 ml of PBST (30 seconds each). Each blot is transferred to a Plexi-glass wash vessel having 30 ml of was solution (PBST). The blots are rotated on a Gyro-shaker (Speed-˜100 RPM) and washed 6 times with wash solution.

For conjugate binding, a 1:10000 dilution of Neut-avidin-HRO conjugate in PBS is prepared with 10 ml of conjugate solution for each blot. The 10 ml of conjugate is dispensed in the Omni tray and rocked for 45 minutes or a vertical rocker. To analyze the initial or “primary” binding of gene products to the array, a positive IgG blot is washed twice with wash solution and incubated with 10 ml of 1:100,000 dilution of goat anti-mouse-IgG HRP in the Omni-tray while on the vertical rocker. After 45 minutes, the test blots and positive IgG blots are rinsed twice with PBST (2×30 seconds). The blots are placed in Plexibox wash vessel containing 30 ml of wash solution (PBST) and washed 6 times with wash solution. A chemiluminescent substrate is prepared by mixing equal volumes of the two solutions in a 50 ml polypropylene tube. About 7 ml of substrate solution are prepared for each immunoblot. The solutions are mixed in a dark room to avoid the light. Seven ml of substrate solution are dispensed in an Omni-tray and the immunoblot is placed in the tray, followed by incubation in the dark by rocking for 5 minutes on a Vertical rocker. After 5 minutes of incubation, the blots are placed in a plastic sheet cover and the sheet is placed in an x-ray cartridge. The film for various progressive times to gauge the appropriate exposure time (from few minutes to 30 minutes) to achieve dark spots without complete saturation of the spots. Alternatively, a CCD camera is used to capture the reactivity of each antibody in the array.

### EXAMPLE 5

### Disease specific antibody arrays—lung carcinoma

Carcinogenesis is a multistage process which is characterized by a cascade of molecular events. Experimental induction of tumors in animals and neoplastic transformation of cultured cells by chemicals have helped the identification of a number of molecular events that initiate the tumor process as evidenced by the activation of protooncogenes and the inactivation of tumor suppressor genes (Yuspa, 1997; Denissenko,1996; Kratzle, 1996; Rodenhuis, 1992). To define risks, assess factors underlying this pathology, and provide preventive measures, it is necessary to evaluate and classify the effects of exposure to various classes of carcinogens.

Carcinogenic agents induce nucleotide alterations which result in the modification of the cellular pattern of gene expression. These changes may ultimately spread to different cell types and tissues. Consequently, the monitoring of a large number of gene products from the same type of cell or tissue, before and after exposure to toxic agents would allow the correlation of gene expression deregulation with the nature of the toxic agent, and its effects on cell physiology.

Humans are exposed to environmental carcinogenic agents mainly through food, water and air. Indeed, lung cancer is the leading cause of cancer death in the U.S and in industrialized countries (Hecht, 1997). Smoking by itself causes at least 85% of cases of lung cancer. Therefore, the present invention allows for the study of the effect of carcinogenic agents on lung cells by monitoring gene product expression profiles of up to 1000 gene products and possibly several thousand by using the antibody array technology of the present invention. This study includes the following:

Cigarette smoke contains at least 40 chemical compounds that are carcinogens in animals (Hecht, 1997), of which polynuclear aromatic hydrocarbons (PAH) such as benzo[a]pyrene (baP), and 4-(methylnitrosamino)-1-(3-pyridyl)-1-butanone (NKK)-are the most important. Metals compounds, such as those containing nickel and chromium, are also present in cigarette smoke. All of these compounds have been show to induce tumors of the lungs in laboratory animals (Hecht, 1997). Some of them require metabolic activation to form reactive intermediates, which will bind to DNA and initiate the carcinogenic process by formation of DNA adducts (Yuspa, 1997; Hecht, 1997).

DNA damages induced by carcinogens can cause ultimately base mispairing or small deletion, leading to missense or nonsense mutations, as well as chromosome break and large deletions. If these damages remain unrepaired in critical regions of genes, they can cause permanent deleterious changes in oncogenes, tumor suppressor genes, and other important cellular growth control. The result will be the derangement of normal growth control processes resulting in cancer.

Conventional techniques utilizing DNA amplification and hybridization to oligonucleotide probes, direct sequencing methods, RTPCR, northern blot, in situ hybridization and immunohistochemistry have been utilized to determine transcript levels as well as presence of gene mutations in specimen samples from lung cancer, and in cell culture or animal models exposed to carcinogens (Kratzke, 1996; Rodenhuis, 1992; Belinsky, 1996; Shiao, 1998; Dubrovskaya, 1998). Activation of protooncogenes (ras, myc, erbB-2), inactivation of tumor suppressor genes (p53, Rb, p16^{INK4a}) and change in the expression of oxidative stress genes or enzyme that methylates the DNA (DNA-methyltransferase) are examples of genes that were associated with lung cancer in human and in animals (Kratzke, 1996; Rodenhuis, 1992; Belinsky, 1996; Shiao, 1998; Dubrovskaya, 1998). These techniques are however limited to the study of changes in the expression level of transcripts and the mutation or deletion of a small number of genes suspected to be involved in the pathology.

Although several competing technologies can be applied to access gene expression profiles, the technology of the present invention has substantial advantages: a) antibody arrays monitor directly the final gene product (i.e., glycosylated, phosphorylated) as compared to transcripts; b) as tools, antibodies can be utilized to discriminate in high throughput assays which are the receptors, secreted molecules, and/or nuclear proteins; c) antibodies can also be used in high throughput functional assays to identify the biochemical pathways involved in pathological states (i.e., growth inhibition, apoptosis, transduction pathways, hormonal effects, chemotherapy effects and alike); and d) antibodies are commercial products by themselves.

For a disease specific indication such as lung carcinoma, the array can be constructed in several ways. Because cDNA libraries that are derived from particular tissue types and disease states are available, an assembly of cDNAs from a lung cancer cell line can be used to produce the antibodies in an array through a process of DNA immunization as described herein. However, given the extremely high efficiency throughput analytical capability provided by the present invention, a vast array of antibodies derived from DNA sequences in other cell types can also be used. Thus, for the analysis of a specific disease condition, the contents of the array are not limited to antibodies derived from DNA sequences where the limited knowledge regarding such sequences is available. In a disease specific array, a control sample, or population of normal control samples is exposed to the array to determine a database of antibody antigen interactions that may be characterized as reflecting a normal physiological state i.e., a physiological state lacking any manifestation of the disease. As will be appreciated by one of ordinary skill in the art, variations from organism to organism will yield variations in the individual reactivities, however, the number of potential targets available provides the ability to separate the disease state from the normal state. Once the normal values are established, samples from the disease state are exposed to an array, preferably an identical array, for ease of correlation of the individually expressed gene sequences.

When the samples corresponding to a disease state are analyzed, detailed information about the disease state is correlated to the reaction of protein in the sample to antibodies in the array. For example, the presence or absence of disease in and of itself may be detected. Furthermore, with underlying information about the nature of the disease and the nature of the organism from which the sample is derived, enables the identification of specific gene sequences that correlate to the specific disease condition or individual aspects thereof. In particular embodiments, samples from early stage cancer patients may illustrate differential expression of certain genes that are not differentially expressed in later stages of the disease. Similarly, as the disease state becomes more progressed, different differential gene expression profiles may be exhibited, e.g., gene sequences and gene products that are particular for vascularization of a tumor or the metastatic spread of tumor cells throughout the body. In each case, practice of the invention enables one to identify the differential expression of gene sequences that comprise both the causative agents of the underlying disease state, as well as physiological events that are components of the overall biological cascade resulting from any disease state or exposure to stimulus. Once the differential expression profiles are obtained, the gene sequences and corresponding gene products can be identified and further studied for use as markers, as well as diagnostic or therapeutic products.

### EXAMPLE 6

### Differential Gene Product Expression Profiles in Normal, UVB-Irradiated Melanocytes, and Malignant Melanoma Cells.

To analyze differential gene expression in response to external stimulus, an initial screening of antibody binding to normal melanocyte gene products are performed by exposing a cell extract to the antibody array. The selected antibodies used to construct the array may or may not be specially selected for the indication. In either event, normal cell extracts are exposed to an array and the specific binding events are determined. Then, cell extracts from UV-B-exposed melanocytes and malignant melanoma cells are exposed to a separate array, preferably having the same orientation and content as the original control.

A preliminary experiment is needed to determine the dose of UVB (40-2000 J/m²), as single or two fractionated doses, 12 hours apart, that causes a growth suppression of melanocytes without being lethal (Vogt, 1997; Pedley, f996). The UV lamp (Stratagene or equivalent) used has a continuous spectrum of UVB light with a peak at 312 nm. The emission of UVC is negligible (<290 nm). About 15% of the total energy is emitted in the UVA range (320-400 nm). Mean flux rate is 20 J/s.

Human newborn melanocytes are grown in 100-mmm dishes to 70-80% confluence over 8 days. On day 8, cells are rinsed with sterile PBS; then the melanocytes are irradiated through a thin layer of PBS with one or two fractionated doses of UVB, 12 hours apart, as determined in the above experiment. After the last irradiation, the PBS is replaced by fresh media, and 12 hours later cells are harvested to prepare protein extracts as described in Medrano (1995).

Malignant melanoma cell lines with various metastatic capacities (i.e., SK-MEL-2, RPMI-70951, WM-115, or C32); available from ATCC) are grown to 80% confluence and similarly harvested to prepare protein extracts. Protein extracts from each are exposed to the antibody array as described above. The comparative analysis of the arrays is performed as follows:

The screening of the antibody arrays are performed by analysis of protein extracts from melanocytes using the following steps:

1) Equal amounts of cell protein extracts (1 mL/spot from a protein concentration of 100 mg/mL) from cells are spotted onto an 8×10 cm size nylon membrane.

2) Unbound sites are saturated with 5% non-fat dry milk in Tris-Tween saline buffer.

3) Diluted mouse antisera (1 ml/dot from an arbitrary sera dilution of 1:1000) are spotted on membrane-bound protein extracts as described precedently (Smith, 1984).

4) Membranes are incubated with secondary antibodies specific to mouse IgG immunoglobulins coupled to peroxidase or alkaline phosphatase, and antibody/antigen complexes are detected by colorimetry, or chemilumiscescence.

5) The results are quantified using a commercially available digital imaging system and appropriate software for antibody array analysis. Spot intensities generated by each antiserum are compared to negative and positive control antisera. Those antisera that generate spots with intensities at least 4-5 times greater than background (negative control) and greater than or equal to positive controls will be analyzed in comparison with the cell extracts of UV exposed and malignant melanoma cells.

Ultraviolet exposure of normal and malignant cells is only one example of the varying stimulus or conditions that can be applied to cells to conduct gene expression profiling at the protein expression level. For example, using the same protocol, the gene expression of varying cell types can be analyzed following exposure to hormones, growth factors, bioactive chemicals generally, drugs, especially chemotherapy compounds, and virtually any toxin or agent whose effect on cell growth or metabolism and the underlying gene expression is of interest.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A method to create and use an antibody array to analyze gene expression comprising:
producing antibodies by immunizing a murine animal with a polynucleotide sequence to yield antibodies that bind with an expression product of the polynucleotide sequence, arranging aliquots of the antibodies in discrete areas of a solid support to yield an antibody array, exposing the antibody array to a sample containing an antigen to react the antigen in the sample with the antibodies of antibody array, and correlating binding reactions between antigen in the sample and antibodies in the antibody array with expression of the polynucleotide sequence.

2. The method of claim 1, wherein the antibodies are comprised of monoclonal antibodies obtained by hybridoma derived from the murine animal.

3. The method of claim 1, wherein the antibodies are comprised of polyclonal antibodies.

4. The method of claim 1, wherein the antibodies are a mixture of isotypes.

5. The method of claim 1, wherein the antibodies located at the discrete areas of the solid support are comprised of polyclonal IgG antibodies obtained from immunization of a murine animal with the polynucleotide sequence encoding the antigen.

6. The method of claim 1, wherein the discrete areas of the solid support contain murine sera.

7. The method of claim 1, wherein the step of arranging aliquots of the antibodies is comprised of using between at least 100 different antibodies and 10,000 different antibodies located at the discrete areas of the solid support.

8. The method of claim 1, wherein the step of exposing the antibody array to the sample containing antigen is comprised of contacting the antibody array with a protein derived from human cells.

9. The method of claim 1, wherein the step of correlating binding reactions is comprised of identifying A SET OF polynucleotide sequences expressed in a biological source of the sample.

10. The method of claim 1, wherein at least 10% of the discrete areas of the array are comprised of aliquots of homogenous antibodies.
